(19) United States
(12) Patent Application Publication
(10) Pub. No.: US 2008/0249514 A1
(43) Pub. Date: Oct. 9, 2008

(54) Method and Apparatus for Multi-Step Correction of Ophthalmic Refractive Errors

(76) Inventors: Kristian Hohla, Vaterstetten (DE); Gerhard Youssefi, Landshut (DE)

Correspondence Address: Bausch & Lomb Incorporated, One Bausch & Lomb Place, Rochester, NY, 14604, US

(21) Appl. No.: 12/109,801

(22) Filed: Apr. 25, 2008

Related U.S. Application Data
(63) Continuation of application No. 10/110,891, filed on Dec. 23, 2002.

(30) Foreign Application Priority Data
Oct. 21, 1999 (DE) 19950789.9
Mar. 23, 2000 (DE) 10014481.0

(51) Int. Cl. A61F 9/008

(52) U.S. Cl. 606/5; 128/898

(57) ABSTRACT

A technique of refractive eye correction employs multiple steps to correct refractive errors in the eye. In the first step, gross decentrations of the refractive error are corrected, allowing the subsequent steps to be relatively symmetric in their treatment profile. Then, the eye's refractive error is again measured, and a subsequent treatment is applied for the remaining error. The overall treatment is thus completed in two or more steps.

### CROSS REFERENCE TO RELATED APPLICATIONS

This application is a continuation of, and claims priority to, parent application U.S. Ser. No. 10/110,891 entitled Method and Apparatus for Multi-Step Correction of Ophthalmic Refractive Errors filed on Dec. 23, 2002, and to PCT Application Number PCT/EP00/10377 filed on Apr. 26, 2001, German National Application Number 10014481.0 filed on Mar. 23, 2000, and German National Application Number 19950789.9 filed on Oct. 21, 1999, the subject matters of which are incorporated by reference herein in their entireties.

### TECHNICAL FIELD

The invention generally relates to refractive correction systems, and more particularly, to a technique for correcting refractive errors in multiple steps.

### BACKGROUND ART

The field of ophthalmology for the past number of years has seen great strides in the development of refractive treatments intended to correct the vision of the eye. These techniques have evolved from the earlier radial keratotomy technique, in which slits in the cornea allowed the cornea to relax and reshape, to present techniques including photorefractive keratectomy (“PRK”), anterior lamellar keratectomy (“ALK”), laser in situ keratomileusis (“LASIK”), and thermal techniques such as laser thermal keratoplasty (“LTK”). All of these techniques strive to provide a relatively quick but lasting correction of vision.

At the same time, the diagnostic tools to determine what correction is needed have also advanced. A variety of new topography systems, pachemetry systems, wavefront sensors, and overall refractive error detection systems can detect not only the amounts of myopia, hyperopia, and astigmatism, but also, higher order aberrations of the eye, shapes and thickness of eye components and a host of diagnostic information for therapeutic use such as correcting or modifying the refractive properties of the eye; i.e., creating better vision. These diagnostic systems and techniques have the potential for permitting correction of both the fundamental and higher order defects, especially when used with even more refined refractive correction techniques, with the possibility that vision correction to better than 20/20 will someday be the norm.

A number of these higher order defects can be either induced by unsuccessful refractive treatment or can be inherent problems with the eye. For example, both radial keratotomy and laser refractive techniques can result in an asymmetric vision correction profile for a variety of reasons. Radial keratotomy can result in an over- or under-relaxation of one portion of the eye relative to the other, whereas laser techniques, especially if not properly centered, can result in a vision correction profile that is off of the optical or visual axis or some other axis of treatment. Advanced laser refractive techniques have in fact been used to subsequently correct for these off axis or otherwise asymmetric refractive errors. Moreover, photorefractive laser surgery for correction of myopia, hyperopia and/or astigmatism has been shown to induce higher order defects, both symmetrical such as spherical aberration and asymmetrical such as coma.

### SUMMARY OF THE INVENTION

According to one feature of the invention, a technique is provided for correcting for asymmetric errors, i.e., defects that vary in magnitude about a defined reference axis, of the eyes in more than one step. First, one or more of a variety of diagnostic tools, such as, preferably a surface elevation-based topography system, or, alternatively a wavefront sensor, is employed to determine the refractive correction necessary to correct an off-axis (decentered) or otherwise asymmetric refractive error. Then, a treatment profile is calculated which does not necessarily fully correct vision, but rather converts, via partial correction; the off axis and/or asymmetric error into a relatively symmetric error. Then, the refractive error of the eye is again examined, and a follow-up treatment is performed to take the then partially corrected vision to fully corrected vision by correcting the residual symmetric defect.

Sometimes, when an asymmetric error is treated, the actual refractive results that do not necessarily match the predicted results. This can be for a variety of reasons. For example, an irregular thinning of the cornea can cause a reshaping of the cornea, which may be difficult to factor into calculations. This may depend upon the healing response, epithelial regrowth, etc. Further, ablation patterns are typically designed based upon a predicted amount of tissue removal per shot, but the actual ablation value can vary. Also, the refractive treatment can affect the tension in collagen fibers in the cornea causing reshaping. By first “pretreating” the eye to convert an asymmetric and/or off-axis error into a relatively on-axis and/or otherwise symmetric error, a more symmetric, and empirically verified treatment profile can then be applied to the eye. The follow-up treatment can occur within a very short period of time after the initial treatment, or can occur a matter of days or weeks later, as limited by physiological or other factors.

It will further be appreciated that the multistep treatment described herein is not limited merely to an asymmetric, then symmetric correction. Obviously, an initial step of “regularizing” a cornea must be followed up on the basis of any biodynamic response observed, which could require an asymmetric treatment also for the secondary treatment. Moreover, the multistep treatment comprises, in an embodiment of the invention, correcting lower order aberrations (Zernike 2^{nd} order) with the primary treatment and higher order aberrations (3^{rd} and higher Zernike order) with the secondary treatment. The general concept of the invention, therefore, is to provide a converging solution to the problem of refractive error correction such that subsequent responses to a treatment decrease which then requires a decreased subsequent treatment and so on.

The treatment steps are referred to as an initial, “centering” treatment and then a follow-up treatment preferably on a computer that calculates courses of treatment for a laser system.

### BRIEF DESCRIPTION OF THE DRAWINGS

FIG. 1 is a block diagram of refractive profiles illustrating steps of a technique according to the invention;

FIGS. 2A-2C are a cut-away profiles of a cornea illustrating steps of a technique according to the invention;

FIGS. 3 is a flow diagram showing steps of a method according to the invention;

FIGS. 4A and 4B are profiles of refractive treatment profiles corrected according to the invention; and

FIG. 5 is a diagram illustrating a typical diagnostic and treatment system according to the invention.

### MODE(S) FOR CARRYING OUT THE INVENTION

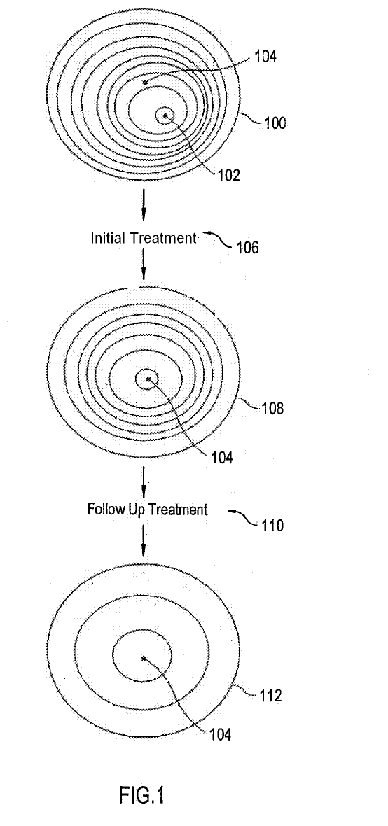

Turning to FIG. 1, illustrated are the steps of one technique implemented according to the invention. Generally, one of a variety of techniques, preferably topographically based, but including others as described below, determines the refractive error profile of the eye. Based on that error, a corresponding partial refractive treatment is then calculated that is sufficient to generally “re-center” and/or symmetrize the remaining refractive error. The treatment is applied, and the remaining refractive error profile of the eye is again measured. Based on this remaining error, a second treatment is calculated and applied to the eye. The initial treatment thus performs the bulk of the decentered off axis, or asymmetric correction, and the subsequent treatment is substantially symmetric.

Referring to FIG. 1, shown is a representation of a refractive profile 100 of a typical eye which can be treated according to this technique. As shown, it includes a refractive error that has a center 102, which is away from a center 104 of the eye. As used herein, the term “center of the eye” refers to a visual axis of the eye defined typically by fixation and alignment, and corresponding with a measurement axis of the diagnostic or therapeutic device, as is well understood by those skilled in the art. The refractive profile 100 corresponds to a variety of different representations of refractive error in the eye. The profile 100 can correspond to a topography map of a surface topography of the eye provided by a typical topography system. One such system was the ORBSHOT™ by Orbtek, Inc., of Salt Lake City, Utah, which produced a variety of representations of the eye's refractive error, including topography maps and dioptric error maps based on the surface topography of the eye. The profile 100 can also represent the error of the overall optical path of the eye, rather than only the surface. Some systems use algorithmic techniques to derive such errors based on the profiles of various optical surfaces in the eye. One such system is the ORBSCAN II( by Bausch & Lomb/Orbtek, which uses surface elevations and ray tracing to determine refractive errors in the eye. Other systems use direct measurements of such errors, such as the wavefront sensor described in U.S. Pat. No. 5,777,719 to Williams et al. Further, combinations of techniques can be used to determine the refractive error profile 100 and a variety of other techniques can be used.

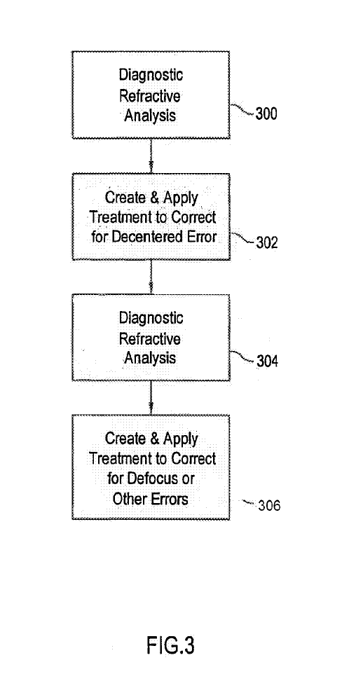

Once this error profile 100 is developed, an initial treatment is developed in a step 106. Creating appropriate treatment profiles from error profiles is well known to the art. Generally, the initial treatment 106 is of a profile that will result in the eye's remaining refractive error being substantially symmetric and on-axis. It need not be perfectly so, because the purpose of the initial treatment is to ensure the subsequent treatment, discussed below, does not have gross volumetric asymmetries. But generally, the initial treatment 106 will be sufficient to remove gross asymmetries. Examples of the initial treatment 106 are discussed below in conjunction with FIGS. 3A-3B. This initial treatment 106 can be developed in a number of ways. Assuming excimer laser surgery is to be performed, for example, a volumetric removal treatment profile for fully correcting the refractive errors of the eye can be developed based on the error profile 100. Then, software can determine a minimum asymmetric treatment profile necessary to yield a remaining treatment profile that is substantially symmetric on the eye. Alternatively, the initial treatment 106 may be more extensive, including a portion of the treatment necessary for the symmetric error correction as well.

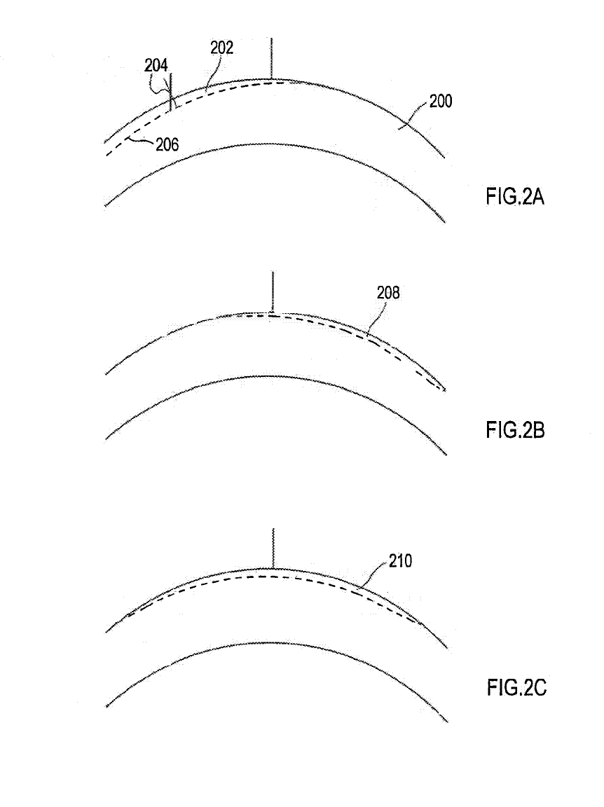

In any case, once this initial treatment 106 is derived the eye is treated, whether by LASIK, PRK, thermal techniques, or any of a variety of other techniques that have been or will be developed. This results in the eye having a new, intermediate refractive error profile 108, which is generally substantially symmetric about the approximate center 104 of the eye. The initial treatment 106 will necessarily have resulted in removal of more tissue on one portion of the eye then the other, as is illustrated in FIGS. 2A-2C below. The intermediate profile 108 is generally symmetric about the axis 104, but may be radially symmetric or axially symmetric. Alternatively, the initial treatment 106 could include correction for astigmatism, yielding a generally radially symmetric profile as the profile 108.

Further, the profile 108 is generally symmetric, but may include higher order, but minor, errors to be corrected, for example, through laser profiling. Again, the point of the initial treatment 106 is to remove the majority of the tissue necessary to generally center and symmetrize the intermediate refractive profile 108. This reduces the effects of gross asymmetries in subsequent treatment; thus, the results of the subsequent treatment become more predictable.

After the initial treatment 106, with LASIK, preferably the flap would be replaced on the eye, which then is allowed to heal—a relatively short process. Alternatively, the eye can be immediately analyzed to determine the results of the LASIK treatment, perhaps adjusting the analysis based on known effects of edema, or swelling. Then, the eye is again refractively analyzed, again using one of a variety of techniques. At this stage of analysis, the same or a different refractive diagnostic tool can be used as is used in diagnosing the initial profile 100, and the tool can even be built into the laser treatment station.

A follow-up treatment 110 appropriate to correct the intermediate refractive error profile 108 is derived, and that treatment is then applied, yielding a final profile 112, preferably the perfect profile for perfect refractive correction of the eye, yielding emmetropia. This is centered at the eye's center 104, and although a slight topography is shown, preferably this topography is the topography necessary to yield perfect vision correction.

Turning to FIGS. 2A-2C, illustrated is a side profile view of a cornea 200 illustrating the steps implementing a technique according to the invention. In FIG. 2A, assume the cornea 200 has previously been treated to correct for myopia using a treatment profile 202, but this treatment profile was unfortunately misaligned on an axis 204. This has yielded a cornea surface defined by the line 206, resulting in an off-center refractive profile, such as the profile 100 of FIG. 1. It is this refractive profile 100 which is to be corrected. Turning to FIG. 2B, a tissue removal is calculated to yield a treatment profile that removes a section of tissue 208, which corresponds to the treatment necessary to convent the off-axis refractive profile 100 of FIG. 1 to the on-axis refractive profile 108. Then turning to FIG. 2C, a subsequent portion 210 is removed in the follow-up treatment 110 of FIG. 1, correcting for a remaining amount of myopia.

As discussed in conjunction with FIG. 1, the refractive profile can be defined in a number of ways. For example, the tissue 208 to be removed to FIG. 2B could be that tissue necessary to theoretically yield a symmetric refractive profile defined in terms of cornea elevation. The previously discussed ORBSCAN II® topography system by Bausch & Lomb/Orbtek defines various refractive surfaces in terms of elevation, and can define both surface elevations of the anterior surface of the eye and elevations of the posterior surface of the cornea as well. Other systems define the refractive profile in terms of directly measured corneal curvature instead of surface elevation. Although such systems ultimately measure the same types of topographies, they do so employing different techniques, and each type of system has advantages.

Rather than defining the desired intermediate refractive profile 108 in terms of surface topography, the goal can be to achieve a cornea with a symmetric corneal thickness. For example, it may be desired to make the initial treatment 106 such that the cornea thickness is essentially the same at a predetermined distance from the center of the cornea. This forms a regular cornea thickness rather than a regular anterior surface profile (although the two will typically be similar). But starting from this regular cornea thickness, the eye can then be treated to refractively correct the remainder of the errors and the follow-up treatment 110.

Illustrating the typical steps that would be applied, FIG. 3 illustrates first at step 300 a diagnostic refractive analysis is performed on the eye, then at step 302 the appropriate treatment is applied to correct for the determined decentration and/or asymmetry. The results are then analyzed in a step 304, which can occur minutes, hours, days, or weeks later, and then the further refractive corrections are applied at steps 306.

When an eye requires an irregular treatment profile, the desired result is a symmetric refractive profile, but the very fact that the treatment profile applied is irregular can induce irregularities in the resulting refractive profile of the eye. For example, the thinning of one portion of eye relative to the other can induce its own refractive effects. Thus, the follow-up treatment 110 will generally correct not only myopia or hyperopia, and certain higher order effects, but will also correct for any unpredicted refractive error induced by the initial treatment 106. In any case, the follow-up treatment 110 will typically be far less asymmetric then the initial treatment 106, thus only minimally inducing additional asymmetric refractive error. It is further possible to perform the process in more than two steps, having a further follow-up treatment for slight decentration that may result. This may be indicated for particularly gross asymmetries.

There are other reasons for attempting to create a regular refractive error profile in the initial treatment 106 to be corrected in the follow-up treatment 110. While an excimer laser, for example, can very precisely remove tissue from the cornea, the actual treatment profile necessary to correct for different degrees of myopia, hyperopia, and astigmatism have been found to require adjustment based on empirical results. These adjustments can depend on many factors, such as the amount of correction, and whether a treatment is an initial treatment or a subsequently performed treatment.

Thus the general embodiment of the invention is to obtain a diagnostic measurement of the patient's eye and to make a first-stage treatment preferably to remove or correct gross defects. The eye's response to the surgical trauma, which may comprise merely the flap cut of a LASIK procedure, is observed. Based upon the observation of the biodynamic response, a second-stage of the multi-stage treatment is performed. Again, the biodynamic response is observed and treatment is continued as appropriate or is considered complete. The preferable outcome is a converging solution embodied by a progressively smaller response and/or more complete correction after each treatment stage.

The empirical results of a number of standard types of treatments generally become established over a large number of treatments. For example, in certain circumstances and conditions one may find an ablation rate in corneal tissue of 0.35 microns removed by a 120 mjoule per square centimeter per shot (a variety of rates are possible, however). If one were to assume such an ablation rate, one would typically find that ablation on a PMMA plate with the theoretically calculated profile would yield the theoretically predicted amount of correction for both myopia and hyperopia. In practice on an actual cornea, however, a single, fixed ablation rate may not yield the result predicted based on a uniform ablation rate; instead, the amount of ablation necessary is typically dependent on whether myopia or hyperopia is to be treated, and the amount of treatment. For example, to treat for −6.00 diopters of myopia, instead of assuming the ablation rate of 0.35, one might use a theoretical ablation rate of 0.46 to calculate the treatment profile. Thus, the treatment profile desired would be a standard treatment profile for −6.00 diopters of myopia, but multiplied by 0.35/0.46. Therefore, the actual treatment profile employed would be the equivalent of theoretical treatment for approximately −4.50 diopters of myopia. Put another way, less ablation is needed than is theoretically predicted. On the other hand, to treat for hyperopia, such as +6.00 diopters of hyperopia, an ablation rate of 0.25 microns per shot can be used in the calculation, and thus to treat for hyperopia of +6.00 diopters, one would actually apply an ablation profile that would theoretical yield the result of +8.40 diopters assuming a constant ablation rate. Alternatively, one could assume a fixed ablation rate but instead scale the desired treatment. That is, one could scale down the treatment to be calculated for myopia from −6.000 to −4.50, and scale up the treatment to be calculated for hyperopia from +6.00 to +8.40. Similarly, the amount of under/overtreatment necessary could be quantified as a percentage. For example, it could be empirically determined that for myopia within a particular range, the actual treatment should only be 75% of the otherwise calculated treatment; for hyperopia, perhaps, a 135% scaling factor is appropriate. The point of all this is not a specific empirical treatments that are developed and how they differ from simplified theoretical calculations based on constant ablation rates, but rather the fact that such empirically developed treatments often yield better results than treatments based purely on theory. By placing the eye in a condition for which many previous treatments have been performed—such as myopia or hyperopia with varying amounts of astigmatism—that empirical data and experience can be brought into play.

There are a variety of reasons that the empirical data diverges from the theoretically predicted outcomes. The cornea tissue is made up of collagen fibers, which are under tension. When the ablation “cuts” those fibers, it could allow additional water to be absorbed into the collagen, effecting the resulting ablation profile. The result could also be influenced by the thinning of the cornea, and the resulting “bulging” of the treated cornea. Also, the deviation of actual treatments from theoretical results is important in subsequent ablation treatments. It has been seen that when performing a follow-up ablation on a cornea, far less actual ablation is necessary than would be predicted to achieve a desired result. Therefore, only a portion of the predicted ablation is needed. Typically, this would range somewhere between 40 to 80% of the theoretically predicted amount of ablation needed, and preferably around 60% of the theoretically required ablation.

As additional empirical data is gathered, it can yield ever more precise results and take into account additional variables. For example, the thickness of the cornea, whether the treatment is a “retreatment”, and other variables could eventually be factored into the empirically developed treatment. Further, empirical data may further provide courses of treatment not only for myopia, hyperopia, and astigmatism, but also for higher order errors. But again, by achieving a known “starting point”, that data can be brought to bear.

The overall effect of these differences between the theoretical outcomes and the empirical outcomes is that it is preferable in a two step treatment to employ the initial treatment 106 to yield a resulting refractive error profile 108 for which empirical data is available. Thus, if the initial treatment 106 yields a refractive error profile 108 that, for example, simply requires −2.00 diopters of myopic correction with −1.00 diopter of astigmatism, generally such refractive treatments will have historical, empirical data from which surgeons can draw, thus appropriately adjusting any theoretical ablation profile to yield the actual desired result.

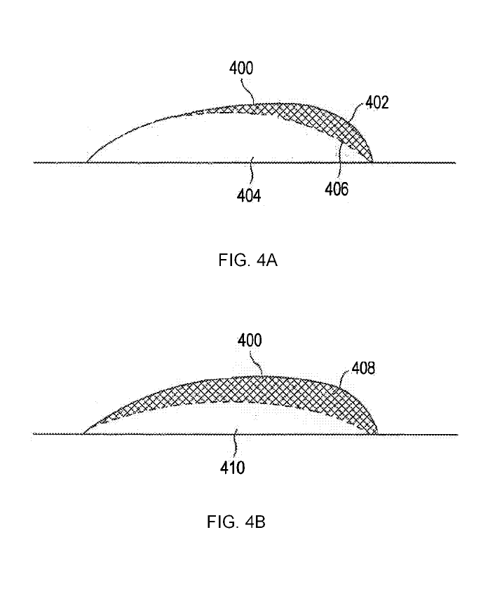

FIGS. 4A and 4B show two alternatives of how to calculate both the initial treatment 106 and the follow-up treatment 110. In FIG. 4A, the preferred approach shown is a cutaway side view of an overall treatment profile 400 derived from the refractive error profile 100 of FIG. 1. This overall treatment profile 400 is exemplary of a course of volumetric removal using a LASIK technique, for example, that would correct for the refractive error profile 100 of an eye. Typically, such treatments have historically been applied in a single step. As discussed above, according to the techniques of the invention, however, the treatment is applied in two steps, the first being a course of treatment 402 illustrated by the crosshatched area, and the second being a generally symmetric course of treatment 404. To develop this two-step approach, first the necessary refractive profile 400 is developed based on the refractive error profile 100. Then, in FIG. 4A, software determines a largest symmetric profile of tissue removal 406 that could be removed given the overall profile 400. Then, that treatment 406 is “subtracted out” of the treatment profile 400, yielding the appropriate treatment profile 402 to correct for the gross decentration and other asymmetrics. Then, the profile 402 is removed in the initial treatment 106, the eye is again refractively analyzed, and then a follow-up treatment provided for what remains. As discussed above, it will be appreciated that this follow-up treatment generally be of a similar profile as the profile 404, but not necessarily identical, as they eye may have slightly changed shape as a result of the initial treatment 106 in which the profile 402 was removed.

FIG. 4B illustrates yet another, alternative approach starting from the same profile 400, but in this case removing a larger amount of tissue in an initial profile 408. In this case, a symmetric treatment profile 410 is calculated, but not to be the maximum symmetric treatment that could be applied to the eye. Instead, lesser symmetric treatment profile 410 is subtracted from the overall treatment profile 400. Then, the initial treatment 106 is provided using the profile 408.

In this approach of FIG. 4B, the initial treatment 106 can yield a result that is “closer” to the final desired result, but still leaving enough of a “cushion” that more or less tissue can be removed than would otherwise be predicted by the treatment profile 410. That is, if the entire treatment 400 was initially performed on the eye, and then a follow-up treatment 110 was applied, extra tissue would typically be removed that would otherwise not have to be removed employing the two-step approach. Leaving the symmetric under-correction represented by the intermediate refractive profile 108, the follow up treatment 110 removes a precise necessary amount of tissue yielding a predictable result. A problem with this approach, however, is that the greater the amount of tissue removed in the initial treatment 106, the greater the unpredictability of such treatment, making it more difficult to yield a symmetric refractive error profile as the refractive error profile 108 for the follow-up treatment 110.

In sum, while even symmetric treatments for conditions such as myopia, hyperopia, and astigmatism typically yield refractive end results that differ from the predicted result, these differences are predictable based on empirical data. That is, based on corneal thickness, surface profiles, previous treatments, and other parameters, doctors can predict how much to “adjust” the actual course of refractive treatments to yield the optimal end result. So employing techniques according to the invention, as illustrated in FIGS. 4A and 4B, the eye is first treated such that it still has a refractive error remaining, but this refractive error is such that it can be very predictably treated. The first step thus eliminates gross asymmetries in the eye, yielding a generally symmetric profile (although still with some higher order irregularities and some low order irregularities) and then the residual, preferably symmetric refractive error profile can be very predictably treated yielding the desired end result.

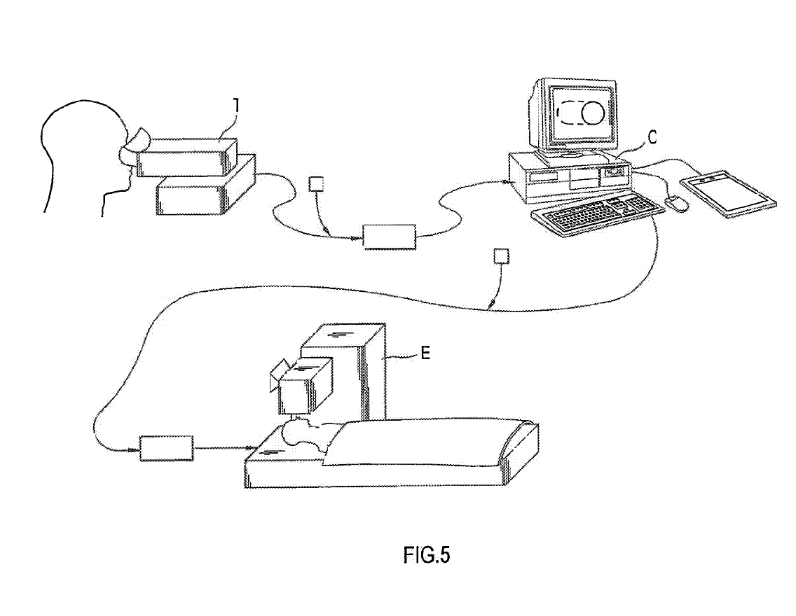

Turning to FIG. 5, shown as a typical combination of a topography system T, a computer system C, and an excimer laser eye surgery system E, coupled to perform techniques according to the invention. Such a system is described, for example, in U.S. Pat. No. 5,891,132 to Hohla, which is hereby incorporated by reference. Topography system T can be one of the above-described systems, or other refractive diagnostic system and the computer system C is generally a personal computer compatible with the IBM PC by International Business Machines, preferably including a fairly high-powered processor. The laser system E can be a variety of systems, including the Keracor 217 by Technolas GmbH of Dornach, Germany.

Generally, the computer system C runs the software which develops a course of treatment based on parameters provided by the physician as well as data from the topography system T. It can employ a variety of algorithms, generally depending on the type of excimer laser system E. If the excimer laser system E employs a relatively large fixed spot size, for example, algorithms described in PCT Application Serial No. PCT/EP95/04028 can be used to develop a course of treatment based on an initial refractive profile and a desired refractive profile. Of course, a variety of laser systems and algorithms provide for treatment of irregular refractive errors, and software suitable for a particular laser system should be employed to develop the refractive profiles as illustrated in FIGS. 4A and 4B.

As will be appreciated, the technique can employ a variety of systems, such as an excimer laser system, a thermal system, radial keratotomy, or related systems, and employ a variety of diagnostic tools, such as a surface topography analysis system, a wavefront analysis system and the like.

The foregoing disclosure and description of the preferred embodiment are illustrative and explanatory thereof, and various changes in the components, circuit elements, circuit configurations, and signal connections, as well as in the details of the illustrated circuitry and construction and method of operation may be made without departing from the spirit and scope of the invention.

We claim:

1. A method for photoablatively treating an eye having a refractive error, comprising the steps of:
determining the refractive error of the eye;
determining an initial treatment plan that partially corrects the refractive error;
performing the initial treatment plan;
determining a residual refractive error of the eye;
determining a follow-up treatment plan that substantially corrects the residual refractive error; and
performing the follow-up treatment plan.

2. The method of claim 1, wherein the step of determining the refractive error of the eye involves determining an asymmetric refractive error of the eye and, wherein the step of determining a residual refractive error of the eye involves determining a residual symmetric refractive error of the eye.

3. The method of claim 2, wherein the step of determining an initial treatment plan involves determining a minimum asymmetric treatment profile necessary to yield a residual treatment profile that is substantially symmetric on the eye.

4. The method of claim 2, wherein the step of performing the initial treatment plan comprises locating the residual symmetric refractive error about an approximate center of the eye.

5. The method of claim 1, wherein the step of determining the initial treatment plan involves determining an initial treatment plan that substantially corrects an asymmetric refractive error of the eye.

6. The method of claim 5, wherein the step of determining the residual refractive error of the eye involves determining a residual symmetric refractive error of the eye.

7. The method of claim 6, wherein the step of determining the follow-up treatment plan involves determining a follow-up treatment plan that substantially corrects the residual symmetric refractive error of the eye.

8. The method of claim 1, wherein the step of determining the initial treatment plan involves determining an initial treatment plan for an asymmetric refractive error of the eye.

9. The method of claim 8, wherein the step of determining a residual refractive error of the eye involves determining a residual asymmetric refractive error of the eye.

10. The method of claim 9, wherein the step of determining the follow-up treatment plan involves determining a follow-up treatment plan that substantially corrects the residual asymmetric refractive error of the eye.

11. The method of claim 10, further comprising determining a remaining residual symmetric refractive error of the eye after performing the follow-up treatment plan and further performing another follow-up treatment plan that substantially corrects the residual symmetric refractive error of the eye.

12. The method of claim l wherein at least one of the steps of determining further comprises the step of making a topographic-based measurement of the eye.

13. The method of claim 1, wherein at least one of the steps of determining further comprises the step of making a wavefront-based measurement of the eye.

14. The method of claim 2, comprising determining the asymmetric refractive error by a topographic technique and determining the symmetric refractive error by at least one of a wavefront sensing technique and a topographic technique.

15. The method of claim 1, wherein at least one of the performing steps comprises LASIK.

16. The method of claim 1, wherein the step of performing the initial treatment plan is a retreatment of a previously treated eye.

17. The method of claim 1, further comprising observing a biodynamic response of the eye at each stage of inflicting a trauma to the eye as part of performing a treatment plan, and modifying a subsequent treatment plan in response to each preceding biodynamic response.

18. A method for determining a multi-stage photoablative treatment plan for an eye having a refractive error, comprising the steps of
determining an overall refractive treatment profile of the eye to substantially correct an asymmetric refractive error and a symmetric refractive error of the eye;
determining a largest symmetric profile of corneal tissue removal consistent with the overall refractive treatment profile;
subtracting-out the largest symmetric profile from the overall profile so as to yield an initial treatment profile suitable to correct the asymmetric refractive error;
determining a first stage treatment plan for obtaining the initial treatment profile;
determining a follow-up refractive treatment profile suitable to correct a residual refractive error; and
determining a second-stage stage treatment plan for obtaining the follow-up refractive treatment profile.

19. The method of claim 18, wherein the step of determining the follow-up refractive treatment profile involves determining a follow-up symmetric refractive treatment profile suitable to substantially correct a residual symmetric refractive error of the eye.

20. The method of claim l 8, comprising using a topographic-based measurement to determine the overall refractive treatment profile and the largest symmetric profile, and using a wavefront-based measurement to determine the follow-up refractive treatment profile.

21. The method of claim 18, wherein the residual refractive error includes one of myopia with or without astigmatism and hyperopia with or without astigmatism.

22. A method for determining a multi-stage photoablative treatment plan for an eye having a refractive error, comprising the steps of:
determining an overall refractive treatment profile of the eye to substantially correct an asymmetric refractive error and a symmetric refractive error of the eye;
determining a symmetric profile of corneal tissue removal that is less than a maximum symmetric profile of tissue removal consistent with the overall refractive treatment profile;
subtracting-out the symmetric profile from the overall profile so as to yield an initial treatment profile suitable to correct the asymmetric refractive error;
determining a first stage treatment plan for obtaining the initial treatment profile;
determining a follow-up refractive treatment profile suitable to correct a residual refractive error; and
determining a second-stage stage treatment plan for obtaining the follow-up refractive treatment profile.

23. The method of claim 22, wherein the step of determining the follow-up refractive treatment profile involves determining a follow-up symmetric refractive treatment profile suitable to substantially correct a residual symmetric refractive error of the eye.

24. The method of claim 22, comprising using a topographic-based measurement to determine the overall refractive treatment profile and the symmetric profile, and using a wavefront-based measurement to determine the follow-up refractive treatment profile.

25. The method of claim 18, wherein the residual refractive error includes one of myopia with or without astigmatism and hyperopia with or without astigmatism.

26. A method for photoablatively treating an eye having a refractive error, comprising the steps of:
obtaining a topographic-based refractive diagnostic measurement of the eye;
performing a first-stage treatment based upon the topographic-based refractive diagnostic measurement including treating a gross asymmetric refractive error of the eye so as to yield a residual, generally symmetric refractive error;
obtaining a wavefront-based refractive diagnostic measurement of the eye after performing the first-stage treatment; and
performing a second-stage treatment including treating the residual, generally symmetric error.

27. A system for determining a photoablative refractive treatment of an eye having a refractive error, comprising:
a computer system adapted to receive refractive diagnostic eye data indicative of an asymmetric refractive error of the eye and a symmetric refractive error of the eye; and
computer software that, when executed by the computer system, calculates an initial treatment plan that is based upon the refractive diagnostic eye data, and which provides an initial treatment profile that is a corrective profile for the asymmetric refractive error, wherein a residual refractive error is substantially the symmetric refractive error.

28. The system of claim 27, wherein the computer software, when executed by the computer system, calculates a follow-up treatment plan that is based upon the refractive diagnostic eye data and, which provides a follow-up treatment profile that is a corrective profile for the residual substantially symmetric refractive error.

29. The system of claim 28, where the follow-up treatment plan is corrective for one of myopia with or without astigmatism and hyperopia with or without astigmatism.

30. The system of claim 28, further comprising a topographic-based system that provides the refractive diagnostic eye data indicative of the asymmetric refractive error of the eye and the symmetric refractive error of the eye to the computer system.

31. The system of claim 30, further comprising a wavefront-based system that provides the refractive diagnostic eye data indicative of the residual symmetric refractive error of the eye.

32. The system of claim 28, further comprising a wavefront-based system that provides the refractive diagnostic eye data indicative of the asymmetric refractive error of the eye and the symmetric refractive error of the eye to the computer system.

33. A multi-stage photoablative treatment plan for an eye having a refractive error, comprising:
an initial diagnostic measurement that provides an overall refractive error measurement of the eye having an asymmetric refractive error and a symmetric refractive error;
a first stage treatment plan derived from the initial diagnostic measurement that provides an initial treatment profile suitable to correct the asymmetric refractive error of the eye,
a follow-up diagnostic measurement that provides a measurement of a residual symmetric refractive error of the eye;
a second-stage treatment plan derived from the follow-up diagnostic measurement that provides a follow-up treatment profile suitable to correct the residual symmetric refractive error of the eye.

34. The multi-stage photoablative treatment plan of claim 33, wherein the second-stage treatment plan is an empirically-based treatment plan that is different than a theoretically-based treatment plan associated with the diagnostic measurement data.
